(19) United States
(12) Patent Application Publication
(10) Pub. No.: US 2011/0077468 A1
(43) Pub. Date: Mar. 31, 2011

(54) DRUG ELUTING EYELID SPECULUM

(76) Inventor: Paul T. FINGER, New York, NY (US)

(21) Appl. No.: 12/894,568

(22) Filed: Sep. 30, 2010

Related U.S. Application Data
(60) Provisional application No. 61/247,009, filed on Sep. 30, 2009.

(51) Int. Cl. A61B 1/32

(52) U.S. Cl. 600/236

(57) ABSTRACT

An eyelid speculum may include two arms each including a curved engagement member. The speculum may be single use, made of plastic, and coated with an antibiotic or other material.

### PRIOR FILED APPLICATION

The present application claims the benefit of prior filed provisional application 61/247,009, filed Sep. 30, 2010, entitled “Drug Eluting Eyelid Speculum”, incorporated herein in its entirety.

### FIELD OF INVENTION

This invention relates to devices such as eyelid speculums used to hold a person's eyelids open for, for example, ocular injections, surgery, treatment, examination, or other procedures.

### BACKGROUND OF INVENTION

Eyelid speculums for opening or separating eyelids are well known in the field of ocular medicine.

A typical eyelid speculum is shown in FIG. 1. The eyelid speculum of FIG. 1 has two arms biased in a separated position. Each arm has an engagement member to engage an eyelid. A physician may squeeze the arms together and then release the arms, causing the engagement members to be at least partially inserted under the eyelids to engage and separate the eyelids. The engagement members separate the center portions of the eyelids thereby exposing the center of the eye, in particular, the cornea.

Such eyelid speculums are useful for eye surgeries in which the access to the anterior surface (or the central anterior surface) of the eyeball is commonly required.

However, when injecting material into an eye, for example, anti-VEGF agents, antibiotics and steroids, it is also desirable to expose other portions of the eye. Common ocular areas for injection include the inferonasal, inferotemporal, superonasal and superotemporal regions.

### SUMMARY OF THE INVENTION

In one embodiment of the invention, an eyelid speculum may include two arms. Each arm may have a first and second end. The two ends may be connected at the first end of each arm. The eyelid speculum may include an engagement member disposed at the second end of each arm. Each engagement member may be curved and may have a middle and two ends. The ends of each engagement member may be farther from the ends of the other engagement member than the middle of each engagement member is to the middle of the other engagement member. Placement of the engagement members will affect which portions of the eyelids are retracted.

In one embodiment of the invention, an eyelid speculum may include two arms. Each arm may have one free end and one connected end. Each connected end may be connected to the other connected end. The eyelid speculum may include a biasing member to bias the arms in a spaced apart relationship. When a force is applied to the arms, the arms may be pushed together in an orientation along a biasing plane. The eyelid speculum may include a plurality of engagement members to engage an eyelid. At least one engagement member may be disposed on each of the free ends. Each engagement member may be curved in the biasing plane, for example, such that the most adjacent surfaces of the engagement members are convex (e.g., convex towards the pupil).

An eyelid speculum according to an embodiment of the invention may be made out of a polymer, plastic or metal material. The speculum may be lightweight, single-use or single-patient use, and/or disposable. The speculum may be coated with a drug, for example with an antibiotic.

An eyelid speculum according to embodiments of the invention may adequately retract, for example, the peripheral and/or central portions of the eyelid to sufficiently expose the surface of the eye.

In embodiments of the invention, the portions of lid retraction can be modulated based on positioning of the engaging members.

### BRIEF DESCRIPTION OF THE DRAWINGS

Specific embodiments of the present invention will be described with reference to the following drawings, wherein:

FIG. 1 shows a conventional eyelid speculum;

FIG. 2 shows the eyelid speculum of FIG. 1 retracting eyelids;

FIG. 3 is a schematic illustration of an eyelid speculum with coating according to an embodiment of the present invention;

FIG. 4 shows the eyelid speculum of FIG. 3 retracting eyelids according to an embodiment of the present invention, with eluting coating touching all surfaces in contact with the speculum;

FIG. 5 is a flowchart of a method according to an embodiment of the present invention;

FIGS. 6 and 7 are schematic illustrations of an eyelid speculum according to an embodiment of the present invention; and

FIG. 8 shows the eyelid speculum of FIG. 6 retracting eyelids according to an embodiment of the present invention.

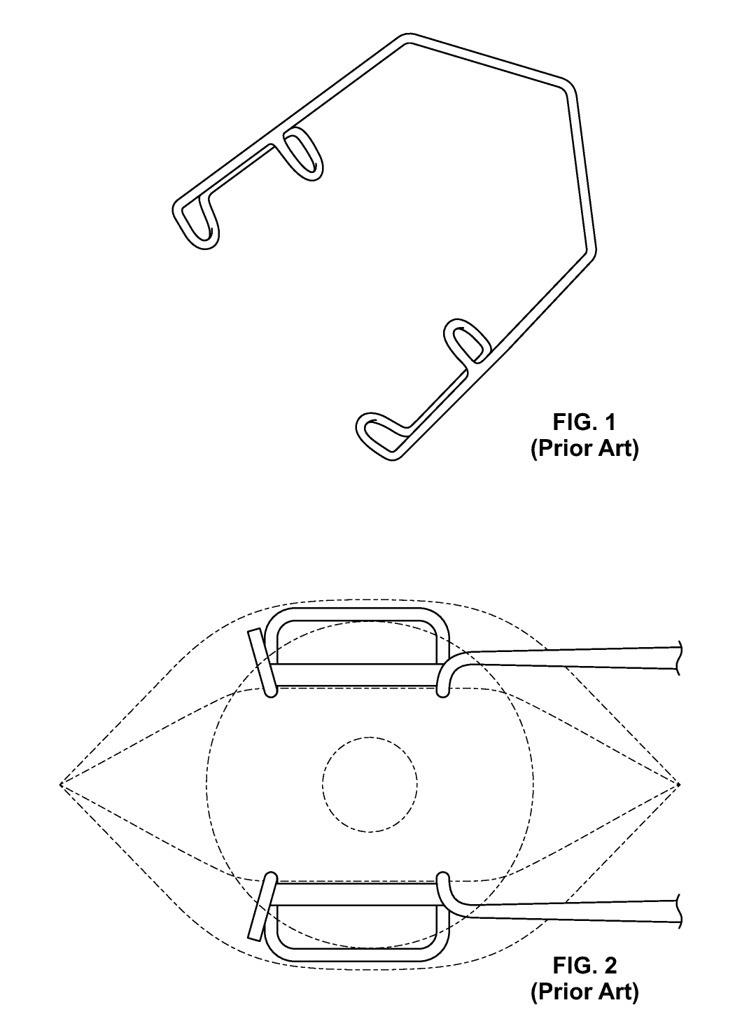

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of embodiments of the invention. However it will be understood by those of ordinary skill in the art that the embodiments of the invention may be practiced without these specific details. In other instances, well-known methods, procedures, components and circuits have not been described in detail so as not to obscure the embodiments of the invention.

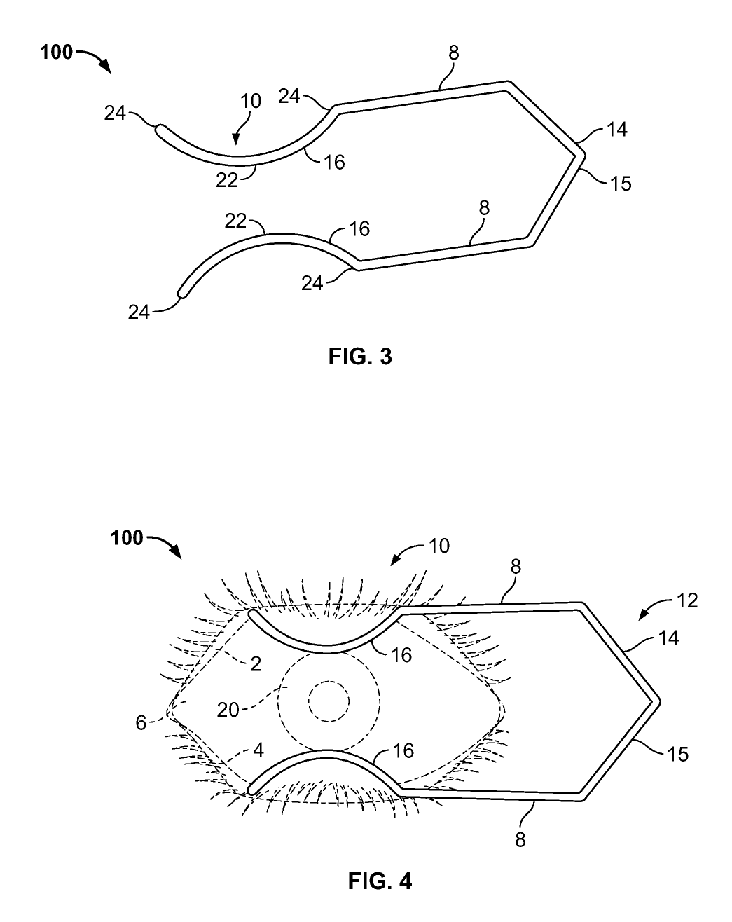

Reference is made to FIGS. 3 and 4, which schematically illustrate an eyelid speculum 100 and the eyelid speculum 100 retracting eyelids 2 and 4, respectively, according to embodiments of the present invention. Eyelid speculum 100 may be used to hold a patient's upper eyelid 2 and lower eyelid 4 open to allow access to an eye or eyeball 6, in particular the peripheral (e.g., nasal and/or temporal) regions thereof, for ocular injections, surgery, treatment, examination, or other procedures. Eyelid speculum 100 may have two elongated arms 8 biased in a separated position. Each arm 8 may have one free end 10 and one connected end 12. The general area of the connected end 12 and possibly part of arms 8 may be a handle or holding portion for a user. A joining member 14 may join arms 8 and may be located at connected ends 12 or alternatively, in the center or anywhere along arms 8. A biasing member 15 may bias arms 8 apart from each other. Biasing member 15 may simply be a part of arms 8 or joining member 14, or may be a separate structure, e.g., a separate spring. The elements that engage the eyelid (engaging members) location can be modulated to preferentially retract those portions of the eyelid.

Eyelid speculum 100 may be used by a person, such as a physician, grasping arms 8, and forcing or squeezing arms 8 towards each other so that they bend, causing engagement members 16 to move towards each other. Arms 8 may move together in an arc or direction along a biasing plane (the plane of view in FIGS. 3 and 4). Eyelid speculum 100 may then be placed near a patient's eye in the proper orientation, e.g., as shown in FIG. 4. Once placed, the physician may remove the force and release arms 8 causing the engagement members 16 to move away from each other, and toward their original relaxed positions. As arms 8 separate, at least a portion of engagement members 16 may slip under eyelids 2 and 4 to engage and separate eyelids 2 and 4.

An engagement member 16 may be disposed on each of free ends 10 of eyelid speculum 100. Engagement members 16 may be adapted to fit at least partially under eyelids 2 and 4 to engage and fixedly retract eyelids 2 and 4 in a spaced apart relationship to preferentially reveal portions of the eyeball surface 6.

Engagement members 16 may be curved (e.g., curved inward, towards the direction arms 8 travel when device 100 is compressed) and may have a middle section 22 and two ends 24. The curvature of engagement members 16 may determine which parts of the eyelids are retracted. When arms 8 are pressed towards each other, each end 24 of each engagement member 16 may be farther from the end 24 of the other engagement member 16 than middle section 22 of each engagement member 16 is to middle section 22 of the other engagement member 16. Engagement member 16 may have a convex shape and two ends 24 of each engagement member 16 point away from two ends 24 of the other engagement member 16 and towards the adjacent eyelid 2 or 4. In this embodiment, when arms 8 are released, the laterally peripheral ends 24 extend further in the direction of the adjacent eyelid 2 or 4 than middle section 22, which is positioned relatively farther from each adjacent eyelid 2 or 4. Accordingly, ends 24 will apply a greater force to the respective laterally peripheral regions of eyelids 2 and 4 than middle sections 22 will apply to the laterally middle region of eyelids 2 and 4.

Alternatively engagement members 16 may have adjacent or closest surfaces that may be convex in the biasing plane (i.e., the plane in which arms 8 move towards each other). In this embodiment, the distance between adjacent surfaces of engagement members 16 increases as the lateral position from the pupil increases in the biasing plane. In this way, adjacent surfaces of engagement members 16 are curved away from each other and away from the center of eyeball 6, e.g., pupil 20. The convex shape of engagement members 16 causes a greater biasing force to be applied thereby at the lateral periphery of eyelids 2 and 4 as compared to conventional eyelid speculums, which apply the majority of their biasing force at the lateral center of the eyelids. In this way, eyelid speculum 100 reveals a larger surface area of the peripheral portions of eyeball 6 as compared with conventional eyelid speculums.

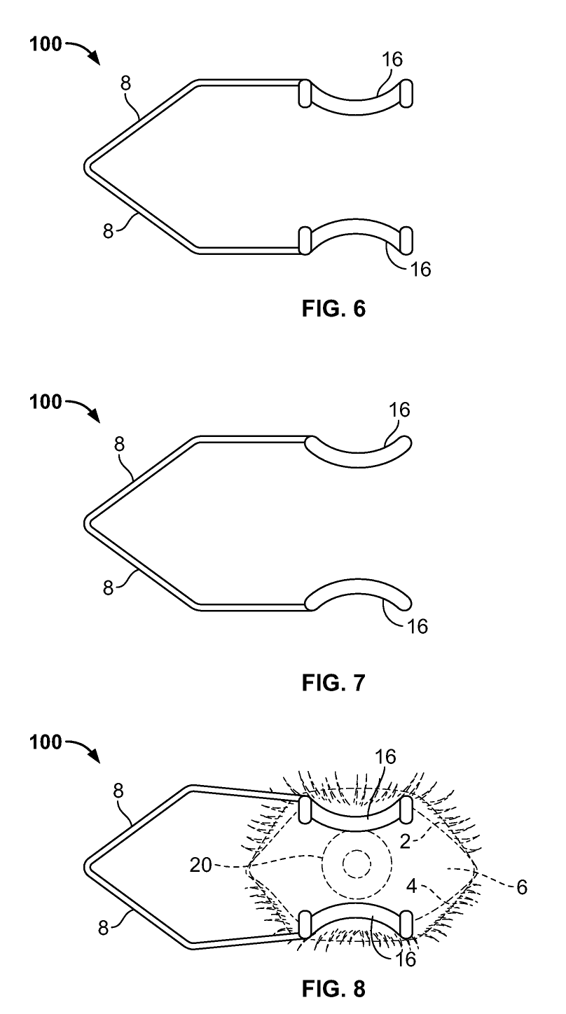

Engagement members 16 may be formed from wire and include hooks (e.g., open leaf), for example as shown in FIG. 6. Engagement members 16 may be made of, for example, a solid sheet or rim of material. Engagement members 16 may include flat surfaces, e.g., blades (e.g., closed leaf), at each end, for example as shown in FIG. 7. Each engagement member may include a hook, blade, or wing disposed at each of two ends of the engagement member. In some embodiment, there may be more surface area to hold a drug or medicine on flat or leaf shaped engagement members.

In one embodiment eyelid retraction may be modulated by physically curving the engaging members or spacing them in such a way as to retract different portions of the eyelid. In one embodiment the arms can be made to only reach the midline, and therefore only retract the lateral portion of the eyelid.

The curvature of engagement members 16 may be continuous, discontinuous or interrupted and may extend across the entire length of engagement members 16 or may be located at one or more sub-section thereof. Arms 8, engagement members 16, joining member 14 and/or other structures of eyelid speculum 100 may be formed integrally, e.g., as one continuous and indistinguishable structure or alternatively, as separate structures, and which may be detachable and/or formed of the same or different materials.

Eyelid speculum 100 may be made out of for example a polymer, plastic or metal material, which may be single-use or single-patient use, and/or disposable. However, any other suitable material may be used. For example, materials may be used which are resilient and have a modulus of elasticity that allows bending of the arms 8 without permanent deformation thereof, and cause the arms 8 to return to their original positions when no force is applied thereto. Alternatively, a screw, spring, syringe pump or other biasing mechanism may be used to bias the arms 8 apart, to hold the aperture of the eyelids at a give distance. Such alternative biasing mechanisms may be located, for example, within or near joining member 14, or on any suitable part of a handle or portion a user holds the device.

Eyelid speculum 100 may be securely held in place due to engagement of eyelids 2 and 4 over a substantial length of engagement members 16. The lateral portion may also rest on the lateral canthal skin. Adhesive, tape, or other supporting devices are not needed to hold device 100 in place between the eyelids, although they may be used.

Eyelid speculum 100 may be coated with an antibiotic or other therapeutic material. Engagement members 16 (e.g., blades or wires) of the device 100 may be coated with one or more therapeutic agents thereby providing a drug-eluding device capable of delivering a therapeutic agent to an area of interest, such as the sclera of the eyeball and inner aspects of the eyelid.

In one embodiment all surfaces of the eyelid speculum 100 are coated or impregnated with one or more therapeutic agents; in other embodiments not all surfaces are coated. In one embodiment, at least one surface of each engagement member 16 is coated or impregnated with one or more therapeutic agents. In one embodiment, all surfaces of the engagement members 16 are coated with one or more therapeutic agents. In one embodiment, surfaces of the portions of the engagement members 16 designated to contact the sclera of the eyeball are coated with one or more therapeutic agents. In one embodiment, surfaces of the engagement members 16 designated to contact the sclera of the eyeball and the lateral and/or nasal aspects of the eyelid are coated with one or more therapeutic agents. In one embodiment, surfaces of the engagement members 16 designated to contact any portion of the eyeball are coated with one or more therapeutic agents. In one embodiment, surfaces of the engagement members 16 designated to contact any portion of the eyelid are coated with one or more therapeutic agents. In one embodiment, surfaces of the engagement members 16 designated to contact any portion of the eyeball and the eyelid are coated with one or more therapeutic agents.

Coating the speculum 100 or the engagement members 16 thereof, with at least one therapeutic agent may prevent infection in patients, provide a general surface disinfectant and sterilizer and/or provide a localized anesthetic. In one embodiment, the coating is continuous. In one embodiment, the coating is discontinuous.

Speculum 100 may be used as a drug or medicine delivery system. A drug may be injected or pumped into a tube or portal on speculum 100, allowed to travel within a hollow internal lumen, and be emitted through one or more openings or fenestrations strategically located on the device. A portal on the handle side (e.g., in the area of connected end 12 or joining member 14) may accept a tube or needle.

Therapeutic agents for coating all or a portion of speculum 100 may provide antibiotic, antibacterial, antimicrobial, antiseptic, antiviral, antifungal, anesthetic or steroid hormonal activities or any combination thereof. In one embodiment, the therapeutic agent is an antibiotic agent. Antibiotics when used herein include all broad and medium spectrum antibiotics, including aminoglycolides, cephalosporins, macrolides, penicillins, tetracyclins and derivatives and mixtures thereof. In one embodiment, the therapeutic agent is an antimicrobial agent. Antimicrobial agents when used herein may include broad spectrum antimicrobial agents that kill gram-positive and gram negative bacteria as well as fungi, yeasts, viruses and protozoa. In one embodiment, the therapeutic agent is betadyne. In one embodiment, the therapeutic agent is an antiseptic. In one embodiment, the therapeutic agent is an antiviral agent. In one embodiment, the therapeutic agent is an antifungal. In one embodiment, the therapeutic agent is an anesthetic. In one embodiment, the therapeutic agent is a steroid. In one embodiment, the therapeutic agent is an antibiotic, antibacterial, antimicrobial, antiseptic, antiviral, antifungal, anesthetic or steroid, or any combination thereof. In one embodiment, the therapeutic agents are betadyne and an antibiotic, antibacterial, antimicrobial, antiseptic, antiviral, antifungal, anesthetic or steroid, or any combination thereof.

Eyelid speculum 100 may be injection molded, or may be manufactured using any desirable method of fabrication.

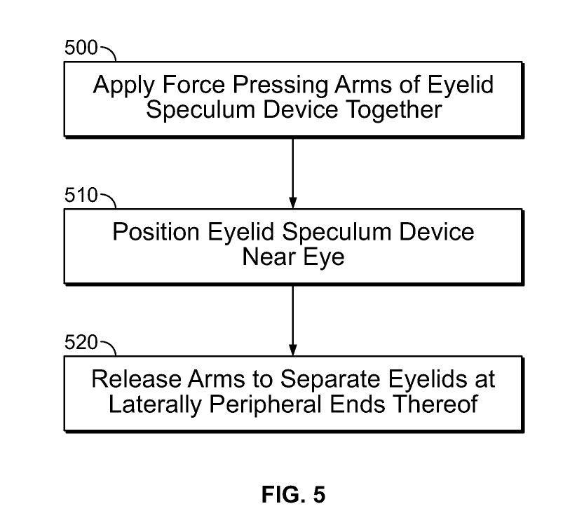

Reference is made to FIG. 5, which is a flowchart of a method according to an embodiment of the present invention.

In operation 500, a physician may apply a force to arms of an eyelid speculum. In a relaxed state, when no force is applied to the arms, the arms are biased in a separated position. Due to the force, the arms are moved towards each other, for example, in a biasing direction in a biasing plane.

In operation 510, while still applying the force, the physician may position the eyelid speculum in a proper orientation, e.g., as shown in FIG. 4.

In operation 520, the physician may release the arms of the eyelid speculum, causing their respective engagement members to move away from each other, and toward their original separated positions. As the arms separate, at least a portion of an engagement members slips under the eyelids to engage and separate the eyelids.

The engagement member may be curved and may have a middle section and two ends. The engagement member may have a convex shape and the two ends of each engagement member point away from the two ends of the other engagement member. When the arms are released, the ends extend towards the adjacent eyelid more so than the does middle section. Accordingly, the ends will apply a greater force to the respective laterally peripheral regions of the eyelids than the middle sections will apply to the laterally middle region of the eyelids.

The eyelid speculum may retract or provide more force to portions of the eyelids in peripheral regions, such as the lateral and nasal aspect, for example relative to portions of the eyelids near the central corneal region, leaving the central corneal portion relatively less retracted. The center of the lid may have less tension than the medial and/or temporal aspects. Alternatively, the eyelid speculum may retract or provide more force to portions of the eyelids near the lateral or the medial aspect alone. When used, portions that are inferonasal, inferotemporal, superonasal or superotemporal may be exposed.

Other operations of orders of operations may be used.

Reference is made to FIGS. 6 and 7, which schematically illustrate an eyelid speculum 100 according to embodiments of the present invention, and to FIG. 8, which schematically illustrates the eyelid speculum 100 of FIG. 6 retracting eyelids 2 and 4, respectively, according to an embodiment of the present invention.

In FIGS. 6-8, eyelid speculum 100 has two connected arms 8. Each arm 8 has an engagement member 16. Engagement members 16 are convex relative to each other in the biasing plane (i.e., the plane of the biasing force or the plane of view in FIGS. 6-8). Each engagement member 16 may include a hook, blade, or wing disposed at each of two ends of the engagement member 16.

In FIGS. 6 and 8, eyelid speculum 100 may be formed from wire, although any metal or plastic may be used, and engagement member 16 may include hooks (e.g., open leaf).

In FIG. 7, eyelid speculum 100 may be formed from metal or plastic and engagement member 16 may include flat surfaces, e.g., blades (e.g., closed leaf), at each end.

Other materials may be used.

Although the eyelid speculum is described herein for use in ocular procedures, the device may be used for other applications. For example, the eyelid speculum may be used to hold an incision in the conjunctiva and Tenon's open during eye surgery. The present invention may be used for any desirable purpose other than an eyelid speculum.

Although the particular embodiments shown and described above will prove to be useful for the many distribution systems to which the present invention pertains, further modifications of the present invention will occur to persons skilled in the art. All such modifications are deemed to be within the scope and spirit of the present invention as defined by the appended claims.

1. An eyelid speculum, comprising:
two arms each having a first and second end, the two ends connected at the first end of each arm;
an engagement member disposed at a second end of each arm, each engagement member curved and having a middle, and two ends, such that the ends of each engagement member are farther from the ends of the other engagement member than the middle of each engagement member is to the middle of the other engagement member.

2. The speculum of claim 1, wherein the engagement members are convex.

3. The speculum of claim 2, wherein the two ends of each engagement member point away from the two ends of the other engagement member.

4. The speculum of claim 1, wherein at least a portion of each engagement member is adapted for insertion under a patient's eyelid.

5. The speculum of claim 1, wherein the eyelid speculum is adapted for single-patient use.

6. The speculum of claim 1, wherein the eyelid speculum is at least partially plastic.

7. The speculum of claim 1, wherein at least a portion of the eyelid speculum is coated with an antibiotic.

8. The speculum of claim 7, wherein the antibiotic is betadyne.

9. The speculum of claim 1, wherein the engagement members are wire.

10. The speculum of claim 1, wherein each engagement member comprises a hook disposed at each of two ends of the engagement member.

11. The speculum of claim 1, wherein each engagement member comprises a blade disposed at each of two ends of the engagement member.

12. An eyelid speculum, comprising:
two arms, each arm having one free end and one connected end, each connected end connected to each other;
a biasing member to bias the arms in a spaced apart relationship, wherein when a force is applied to the arms, the arms are pushed together in an orientation along a biasing plane;
a plurality of engagement members to engage an eyelid, at least one engagement member disposed on each of the free ends, each engagement member being curved in the biasing plane such that the most adjacent surfaces of the engagement members are convex.

13. The speculum of claim 12, wherein the engagement members are convex and each engagement member has two ends, such that the two ends of each engagement member point away from the two ends of the other engagement member.

14. The speculum of claim 12, wherein the eyelid speculum is adapted for single-patient use.

15. The speculum of claim 12, wherein the eyelid speculum is at least partially of plastic.

16. The speculum of claim 12, wherein at least a portion of the eyelid speculum is coated with an antibiotic.

17. The speculum of claim 12, wherein the engagement members are wire.

18. The speculum of claim 12, wherein the engagement members include hooks.

19. The speculum of claim 12, wherein the engagement members are shaped as blades.

20. The speculum of claim 12, wherein at least a portion of each engagement member is adapted for insertion under a patient's eyelid.
